(19) United States
(12) Patent Application Publication
(10) Pub. No.: US 2002/0086027 A1
(43) Pub. Date: Jul. 4, 2002

(54) Composition and method for enhancing immunological memory response

(76) Inventors: Jeanine M. Durdik, Fayetteville, AR (US); Vineeta Bal, New Delhi (IN); Manisha Gupta, New Delhi (IN); R. Suresh, New Delhi (IN); Anna George, New Delhi (IN); Satyajit Rath, New Delhi (IN)

Correspondence Address: HEAD, JOHNSON & KACHIGIAN, E.J. Ball Plaza, Suite 230, 112 West Center Street, Fayetteville, AR, 72701, US

(21) Appl. No.: 09/951,569

(22) Filed: Sep. 10, 2001

Related U.S. Application Data
(63) Continuation-in-part of application No. 09/304,006, filed on Apr. 30, 1999, now abandoned.
(60) Provisional application No. 60/083,803, filed on Apr. 30, 1998.

(51) Int. Cl. A61K 39/395; A61K 31/522; A61K 39/00

(52) U.S. Cl. 424/184.1; 514/263.36

(57) ABSTRACT

A strategy for inducing immune T lymphocytes to respond in such a fashion to their target antigen at first exposure so as to enable them to mount a response of greater magnitude upon any subsequent exposure to the same target is provided. The strategy involves exposure of T cells or animals being immunized to the commonly used phosphodiesterase inhibitor drug, pentoxifylline (POX/PF/Trental), during immune priming. Evidence is provided showing that such exposure leads to enhancement of immune T cells responses upon re-exposure. The strategy is relevant and useful for increasing the efficiency of vaccinations.

### CROSS-REFERENCE TO RELATED APPLICATIONS

Priority is claimed to and this application is a continuation-in-part of U.S. patent application Ser. No. 09/304,006, filed Apr. 30, 1999, which is a continuation-in-part of U.S. patent application Serial No. 60/083,803, filed Apr. 30, 1998, the teachings of which are both expressly incorporated by reference herein.

### STATEMENT REGARDING FEDERALLY SPONSORED RESEARCH OR DEVELOPMENT

The U.S. government may have license rights to the invention under contract with the NIH. The government of India may have license rights to the invention under contract with the NII, Department of Biotechnology, Government of India.

This work was supported by grants to VB and AG from the Department of Science and Technology, Government of India. The National Institute of Immunology is supported by the Department of Biotechnology, Government of India. RS is supported by a grant from the National Institutes of Health, USA (#AI41035). JMD is supported by a grant from the National Institutes of Health, USA (#GM48691).

### FIELD OF THE INVENTION

The present invention relates to a strategy for inducing immune T lymphocytes to respond in such a fashion to their target antigen at first exposure so as to enable them to mount a response of greater magnitude upon any subsequent exposure to the same target. The strategy involves exposure of T cells or animals being immunized to the commonly used phosphodiesterase inhibitor drug, pentoxifylline (POX/PF/Trental), during immune priming. Evidence is provided showing that such exposure leads to enhancement of immune T cells responses upon re-exposure. The strategy is relevant and useful for increasing the efficiency of vaccinations.

### PRIOR ART

For successful protection against various infections, the development of an adoptive immune response is necessary. When the same infection is encountered again by the individual, the immune response specifically directed against target molecules of the infectious organism is significantly different from that seen during the first encounter in being much larger. This ability of the immune system to ‘remember’ past history and respond better to re-infection is called ‘immune memory.’ A peculiarity of immune memory is that once the relevant cellular components of the immune system are ‘sensitized’, they can ‘remember’, or maintain the sensitized state, in the absence of any continuous stimulation by persistence of that infection.

However, in response to an infection, cells of the immune system must not only generate this quiescent state called immune memory, but must also mount a response that is immediately effective and useful in combating the ongoing infection. This pathway of immune cell differentiation must thus lead to the development of ‘effector’ cells. Since effector cells are to be used immediately, a major difference between memory and effector cells is that memory cells must be capable of undergoing proliferation upon re-exposure, while effector cells must be capable of immediate function without proliferating. The immune system, in response to a natural infection, must therefore strike a balance between the generation of memory and effector cells.

However, the requirements are significantly different during vaccination. Vaccination, as commonly understood, is a mock infection, in that is exposes the body to the molecular targets of a given infectious agent without actually causing disease. Thus, there is no immediate need for effector immune responses to vaccination, although most vaccines tend to generate significant levels of effector responses.

In the pathways that regulate the decision-making process of the immune system for striking a balance between effector and memory commitment, there is substantial evidence that the two processes are mutually exclusive, so that stimulating the immune system too hard may lead to the generation of short-lived effector immune cells to the detriment of the generation of long-lived memory immune cells. A dilemma is thus apparent: overstimulating the immune system by a vaccine may be detrimental to the generation of immune memory against it, but understimlulation may also result in poor generation of both effector and memory responses. The solution to this difficulty is the identification and use of immune response modifiers that affect the biochemical pathways governing effector versus memory generation differentially, so as to suppress effector generation and skew the balance in favor of memory generation.

Amongst the cells of the immune system, immune memory is a special attribute of T and B lymphocytes which express antigen-specific receptors on their surfaces. Other cell types such as macrophoges and natural killer (NK) cells do not have this ability. Antibody-making B cells and virus-infected cell-recognizing cytoxic CD8 T cells are heavily dependent on CD4 T cells in order to generate a response, since the CD4 T cells are the main source of interleukin-2 (IL-2), which is a major driver of proliferation in the immune system, and immune cell proliferation is a major prerequisite for a successful immune response. Thus, CD4 T cells are central to any immune response, and their ability to mount memory responses is therefore crucial to the success of most vaccines. We have therefore carried out experiments on CD4 T cell stimulation and memory generation.

Naive CD4 T cells are activated by fragments of their target antigenic protein bound to MHC class II molecules on the surfaces of professional antigen-presenting cells (APCs) such as macrophages, B cells and dendritic cells. These APCs digest proteins into small fragments and these peptide fragments are then bound to MHC class II molecules. T cells need to recognize peptide-MHC complexes, for activation. In addition to this ‘first’ signal from specific peptide-MHC complexes, a T cell needs other signals from APCs for complete activation,—the so-called ‘co-stimulatory’ signals which do not activate T cells by themselves but are essential to complete T cell activation through the first signal. Thus, one mode of controlling T cell responsiveness is based on modulation of the signals that the T cell receives by altering the quality, quantity and kinetics of appearance of both peptide-MHC and costimulatory signals.

To generate an immune response, mature naive peripheral T cells need both cognate antigen-specific and non-cognate costimulatory for optimal activation. If appropriate non-cognate signals are missing, the naive cells may become anergic instead of responding. However, even in the presence of both signals, responding T cells make choices between alternate cytokine profiles (the Th1 and the Th2 groups), as well as becoming either immediate effector cells of quiescent memory cells. In recent years, the role of non-cognate signals,—both cell surface molecules such as the B7 family, and of cytokines like IL-6, IL-10 and IL-12,—in differential T cell commitment to the Th1 and Th2 pathways has been reported. The role of altered peptide ligands in changing effects of cell response has also been documented extensively. However, the regulatory signaling mechanisms controlling the differential commitment to effector versus memory cells have not been as extensively studied. In fact, even phenotypic distinctions between immediate effectors and quiescent memory cells are not reliable, although it is possible to distinguish naive from activated cells.

Following ligation of the TCR-CD3 complex with appropriate peptide-MHC ligands, TCR oligomerization leads to activation of tyrosine kinases such as Ick and fyn followed by ZAP-70. The CD4 or CD8 co-receptors and protein kinases associated with them also contribute to activation. Events further downstream involve the generation of calcium flux and calcineurin activation with transcriptional regulation by both calcineurin-dependent and independent pathways, and induction of transcription factors of the rel and NF-At family involved in regulating may activation-induced T cell genes. Here again there is little information on the differences in signaling that are involved in changing the commitment of T cell differentiation to either the effector or the memory pathway, although differences in signaling events between naive versus formed memory T cells have been studied.

### SUMMARY OF THE INVENTION

We have been studying the effects of pharmacological agents present during T cell priming in vitro on the subsequent secondary ‘recall’ proliferative responses mounted by these primed cells. We have shown earlier that the presence of pentoxifylline (POX) during priming of human T cells in vitro by HLA-mismatched PBMCs results in a decrease in primary proliferative responses but enhanced secondary proliferative responses. POX is a drug used extensively clinically for a variety of inflammatory and vascular disorders. It is a phosphodiesterase inhibitor and can induce increases in intracellular cAMP levels. Its effects have been studied on a wide variety of cell types including T cells, B cells and cells of myeloid lineage. In T cells, it has been shown that the effects may be mediated via modulation of induction of transcriptionally active proteins of the rel family. Here we explore the mechanism of action of POX responsible for the enhancement of secondary or ‘memory’ recall responses. We find that the effect of POX is mimicked by enhancement of cAMP levels in T cells, is independent of IL-2, and is associated with alterations in the degree of apoptosis in the responding T cells.

Naive T cells appear to be primed by specific antigen to differentiate into either effectors or memory cells. We have been analyzing the factors involved in this differential commitment in the priming of allo-responsive human T cells in vitro and have shown that the presence of a phosphodiersterase inhibitor, pentoxifylline (POX3), during priming results in a decrease in the primary response and enhancement in the secondary proliferative response. We now show that the POX-mediated effect can be mimicked by dibutyryl cAMP [dbcAMP]. The secondary response enhancement is due to the effects of POX on the T cells rather than the APCs, since even fixed APCs can prime T cells in the presence of POX. POX affects T cells directly by increasing clonal frequency rather than the burst size of the secondary responders. The known inhibition of IL-2 production by POX is not responsible for this effect, since exogenous IL-2 supplementation does not block it. The presence of POX during priming alters the outcome of T cell activation resulting in a lower frequency of cells expressing IL-2Ra [CD25] and a decrease in their subsequent apoptosis, and this anti-apoptotic effect is consistent with the enhanced commitment of t cells to secondary responsiveness by POX.

In accordance with the present invention, upper and lower ranges of a therapeutically effective dosage or quantity of pentoxifylline is about 100 milligrams to 10 milligrams per kilogram of body weight. This range includes the ED50 for pentoxifylline.

LD50 for the orally administered drug pentoxifylline is about 1385 milligrams per kilogram body weight (Merck Index, 12th Ed., pg. 1228, 1986).

Therapeutically, the drug pentoxifylline is used for a wide variety of indications and the optimal dose used varies from condition to condition. It can be used orally as well as parenterally. The Samlaska and Winfield, Anaya and Espinoza and Capron et al. review articles mentioned above describe the clinical conditions and doses used, and are hereby incorporated by reference.

In accordance with the present invention, a new use for pentoxifylline, a compound (drug) that currently is applied in acute case situations in hospitals, involves the use of pentoxifylline in the enhancement of immunological memory and in an improved vaccination, method, process, protocol, or the like. The new use for pentoxifylline is believed to be a marked, certifiable difference from the way the drug is currently being used. It has been discovered that the application of pentoxifylline at or near the time of immunization enhances the effectiveness of the vaccine.

Also in accordance with the present invention, there has been developed a strategy for enhancing the ability of Tcells to respond to their specific target antigen upon challenge with that antigen by exposing the concerned Tcells to pentoxifylline (or other enhancers of cAMP) during the process of first exposure immunization. Also, there are benefits to using pentoxifylline just before or after immunization.

At present, there are very few approved adjuvants for use in conjunction with vaccines for rendering the vaccine more effective. In accordance with the present invention, pentoxifylline or another enhancer of cAMP is given in combination with the vaccine so that the person or animal being immunized makes a strong secondary immune response. This strong secondary immune response occurs when the person or animal encounters the antigen in the environment.

The pentoxifylline or other enhancer of cAMP should be given either just before, simultaneously with, or after immunization. The administration of the pentoxifylline before or after immunization requires patient compliance and may require a return or additional trip to the vaccination center.

In accordance with the present invention, it has been discovered that low dosages of pentoxifylline provide the desired benefit of inhibiting the primary response and improving or enhancing the memory response to immunization or vaccination, and/or enhancing the magnitude and persistence of immune memory.

Moreover, in accordance with the present invention, pentoxifylline (POX/Trental) can be used as an adjuvant to an immunization or vaccination and thereby enhance the activity of a poor immunogen and provide a therapeutically effective immunization or vaccination.

In accordance with another aspect of the present invention, an immunization protocol involves the use of pentoxifylline and/or other cAMP enhancer as adjuvant before, with, after, before and with, before and after, with and after, and/or before, with and after immunization.

In accordance with a particular example of the present invention, pentoxifylline or other cAMP enhancer or combinations thereof can be used as immunization or vaccination adjuvant any time within a range of five days prior to five days following immunization or vaccination, preferably within a range of three days prior to three days following vaccination or immunization.

Still further, in accordance with the present invention, there has been discovered a new or novel composition, immunization or vaccination including a vaccine, antigen, or immunogen and pentoxifylline or other cAMP enhancer.

Still yet further, there has been discovered a new or novel composition, immunization or vaccination including antigen together with pentoxifylline or other cAMP enhancer and a carrier such as saline solution or distilled water.

In accordance with another aspect of the present invention, there is provided a new or novel composition, immunization or vaccination including pentoxifylline or other cAMP enhancer together with known adjuvants such as Alum plus antigen or immunogen.

Still further in accordance with the present invention, there has been discovered a new or novel composition, immunization, or vaccination including an immune system booster or boosters together with pentoxifylline or other cAMP enhancer, known adjuvants, and antigen or immunogen.

### BRIEF DESCRIPTION OF THE DRAWINGS

FIG. 1 shows that POX (Trental) inhibiting the primary proliferative response, but enhances T cell priming for memory responses. Panel A shows the primary proliferative response against tissue-mismatched stimulators in the presence or absence of 100 ug/ml Trental. Panel B Shows the antigen-specific memory recall responses of cells primed, as in panel A, with various combinations of 100 ug/ml Trental and stimulator cells as shown. Panel C shown the responses of these variously primed cells to the polyclonal T cell mitogen, PHA.

FIG. 2 shows that increasing doses of POX/Trental cause increasing potentiation of T cell memory. Responder cells were primed in the various combinations of stimulator PBMCs and Trental doses as shown and restimulated in the absence of the drug.

FIG. 3 shows a limiting dilution assay of transplantation antigen-specific T cells primed in vitro either in the absence (−PF) or in the presence (+PF) of 100 ug/ml of POX/PF. Panel A shows the calculated frequencies of responding cells in a secondary challenge, while panel B shows the mean±S.E. of the cpm values observed are clonal frequencies in this assay for the −PF and +PF groups.

FIG. 4 shows the results of mice immunized with 1 mg of ovalbumin (OA) in PBS i.p. and treated with either PBS or 1 mg POX/PF per mouse per day from day—1 to day 5 of immunization. Splenic cells were stimulated in vitro either on day 7 or on day 60 after immunization with titrated doses of ovalbumin for 4 days and pulsed with 3H-thymidine for 16 h before being harvested for scintillation spectrometry

FIG. 5 shows the use of PFA for fixing priming APCs had differential effects on primary vs. recall T cell proliferative responses. A, (³H)thymidine incorporation (cpm±SE) by responder PBMCs (1×10⁵/well) fixed variously, as shown. B, the recall response of PBMCs primed in the experiment in A, as described (see Materials and Methods), to irradiated stimulator PBMCs (1×10⁵/well). The data are representative of several independent experiments.

FIG. 6 shows the inhibition of DNA replication during priming and does not prevent significant enhancement of recall responses. A, (³Hjthymidine incorporation by responder PBMCs against irradiated MHC-mismatched stimulator PBMCs at various doses in the presence of varying concentrations of Aph, as shown, B, the effect of the presence of Aph during priming with irradiated stimulator PBMCs on the magnitude of the recall responses to fresh irradiated stimulator cells. The data are representative of five independent experiments.

FIG. 7 shows the Trental inhibiting the primary proliferative responses, but enhancing T cell priming for recall responses of cells primed, as in A, with various combinations of 100 ug/ml of Trental and irradiated allo-PBMCs, as shown. C, the responses of these variously primed cells to IL-2 and D shows their responses to PHA. The data are representative of six independent experiments.

FIG. 8 shows the increased doses of Trental causing increased potentiation of T cell priming. Responder cells were primed in the various combinations of stimulator PBMCs and Trental doses as shown, and restimulated with the same allo-PBMCs in the absence of the drug. The data are representative of three independent experiments.

FIG. 9 shows inhibition of DNA replication during priming does not prevent enhancement of priming by Trental. A, primary responses against irradiated allo-PBMGs in the presence of combinations of 1 ug/ml Aph and 100 ug/ml Trental, as shown. B, the effect of the presence of these drug combinations during priming on the magnitude of the recall responses to fresh irradiated stimulator cells. The data are representative of six independent experiments.

FIG. 10 shows Aph and Trental do not alter the kinetics of the primary alloresponse. The primary proliferative response of responder PBMCs to 2×10⁴ stimulator cells at various days in the presence of various drug combinations is shown. A, responses in the presence of the drugs throughout the 10-day assay period, while B shows the responses when drugs were removed by washing on day 6 (same as for A). The data are representative of three independent experiments.

FIG. 11 shows the presence of Aph and Trental during priming does not alter the kinetics of the recall proliferative response. responder PBMCs were primed with stimulator cells, as in FIG. 5, in the presence or absence of various drug combinations, as shown, or left without priming (none). Recall responses to 2×10⁴ stimulator cells are shown assayed on days 3, 4, and 5. The data are represented of two independent experiments.

FIG. 12 shows the presence of CsA during priming enhancing the priming effect at low doses. A, the primary proliferative alloresponse in the presence of various concentrations of CsA, as shown. B, the recall alloproliferative responses of cells primed in the presence of various combinations of stimulator cells and CsA, as shown. the data are representative of six independent experiments.

FIG. 13 shows that POX enhances secondary response commitment during T cell priming even with fixed APCs. Panel A shows the alloresponse of T cells to g-irradiated (open circles) or PF-fixed PBMCs in the presence (filled squares) or absence (open squares) of POX [100 ug/ml]. Panel B shows recall responses of cells primed with PF-fixed APCs in the presence (filled squares) or absence (open squares) of POX compared to unprimed cells (open circles) when restimulated with g-irradiated PBMCs. Data are shown as mean±SE of triplicate cultures. A representative example of four independent experiments is shown.

FIG. 14 shows that priming in presence of dbcAMP results in a decreased primary proliferative response and enhanced secondary response. Panel A shows a decrease in primary alloresponse with increasing doses (filled circles, 0 um; hollow circles. 10 mM; hollow squares, 100 mM hollow triangles, 1 mM) of dbcAMP. Panel B shows secondary responses of T cells treated with dbcAMP during priming to g-irradiated PBMCs (filled circles, unprimed; filled squares, PBMC-primed; PBMC-primed in the presence of dbcAMP doses as in panel A. Results are shown as mean±SE of triplicate cultures and are representative of five independent experiments. Background counts were 1000-3000 cpm.

FIG. 15 shows that the presence of IL-2 in addition to POX priming during does not suppress the enhancement of secondary proliferative responses. Secondary response of T cells either unprimed (filled circles), primed with allo-PBMCs in the presence (filled squares) or absence (hollow squares) of POX (100 ug/ml), or in presence of IL-2 (3 U/ml; hollow triangles) or of both POX and IL-2 (filled triangles) is shown. Background counts were less than 2000 cpm. The results shown are from one of five independent experiments.

FIG. 16 shows that priming in presence of POX increases the secondary precursor frequency but not the clonal burst size. Panel A shows increase in the frequence but not the clonal burst size. Panel A shows increase in the frequency of T cells either unprimed or primed with allo-PBMCs in the presence or absence of POX (100 ug/ml). Panel B shows mean cpm±SE in positive-scored wells (hatched bars) and in negative-scored wells (hollow bars) for the same three groups. The results are representative of four independent experiments.

FIG. 17 shows that CD25 expression on CD4 cells in primed populations before and after a two day rest is altered by POX. responder cells, either unprimed (panels A and D), or PBMC-primed in the absence (panels B and E), or in the presence of POX (100 ug/ml) (panels C and F were stained for CD4 and CD25 either immediately at the end of six days in culture without any stimuli (panels A,B and C) or after two further rest days in culture without any stimuli (panels D,E, and F). The frequencies of cells in various quadrants are indicated as percentages. The data are representative of four independent experiments.

FIG. 18 shows that CD25 cell frequency is lower to begin with but increases over a rest period in populations primed under POX cover. Data from four independent experiments staining for CD25 were used to calculate the CD25+ cell frequencies (mean±SE) in populations primed with allo-PBMCs in the presence or absence of POX (100 ug/ml) as shown, either immediately after six days of priming (hollow bars) or after two further days of rest (hatched bars).

FIG. 19 shows that the presence of aphidicolin during the two-day rest period after priming does not abrogate the increase in frequency of CD25+ cells in responders exposed to POX during priming. Responders primed with allo-PBMCs in the absence (panels A, B and C) or presence (panels D, E and F) of POX (100 ug/ml) were stained for CD25 immediately after six days of priming (panels A and D), or after rest for two further days in culture in the absence (panels B and E) or presence (panels C and F) of aphidicolin (1 ug/ml). Gates shown for CD25 expression were set from appropriate negative controls (not shown), and the frequencies of CD25+ cells are indicated as percentages. The data are representative of three independent experiments.

FIG. 20 shows that the presence of POX during T cell priming does not alter the pattern of CD95 expression on the CD25+ T cells. Flow cytometric analysis for CD25 and CD95 expression on T cells primed in absence (panels A and C) or presence (panels B and D) of POX (100 ug/ml on day 0 (panels A and B) and day 2 (panels C and D) after priming is shown. Data are representative of two independent experiments.

FIG. 21 shows that the presence of POX during T cell priming decreases apoptosis of primed T cells. T cells primed with allo-PBMCs in the absence (panels A and C) or presence (panels B and D) of POX (100 ug/ml) were stained with anti-CD25 and PI either immediately after the six day priming period (panels A and B) or after 2 days of further rest (panels C and D). The PI staining profiles of gated CD25+ cells are shown. Data represent two independent experiments.

### DETAILED DESCRIPTION OF THE PREFERRED EMBODIMENTS

We intervene directly ‘inside’ the T cell and change the intracellular pathways of signal transduction during antigen exposure, or ‘priming’, so as to enhance the resultant memory response.

The pharmacological intervention we have proposed and used is pentoxifylline (POX/PF/Trental). POX is a vasoactive drug in wide clinical use for vascular, inflammatory and toxic disorders, and it is clear that is affects diverse cell types. Immunologically, POX has been reported to confer significant protection against both myelin basic protein-induced experimental autoimmune encephalomyelitis, and against mercuric chloride-induced arthritis in rats.

We have used two types of experimental systems to demonstrate the effect of POX on immune memory. One system uses human T cells stimulated in vitro with tissue-mismatched transplantation antigens, while the other uses whole animal studies in mice immunized with various antigens under cover of POX treatment.

In the first system, human T cells from peripheral blood mononuclear cells (PBMCs) of one donor (responder) are stimulated with MHC-mismatched PBMCs are irradiated so that they do not respond by proliferation to the responder cell population. In this system, we have used responses to first exposure as an indication of primary responses. When cells stimulated in this fashion are rested in culture and then restimulated with the same stimulator PBMCs, the ‘recall’ response measured are taken as an indication of immune memory. Proliferation is measured by the incorporation of radioactive tritiated thymidine in dividing cells.

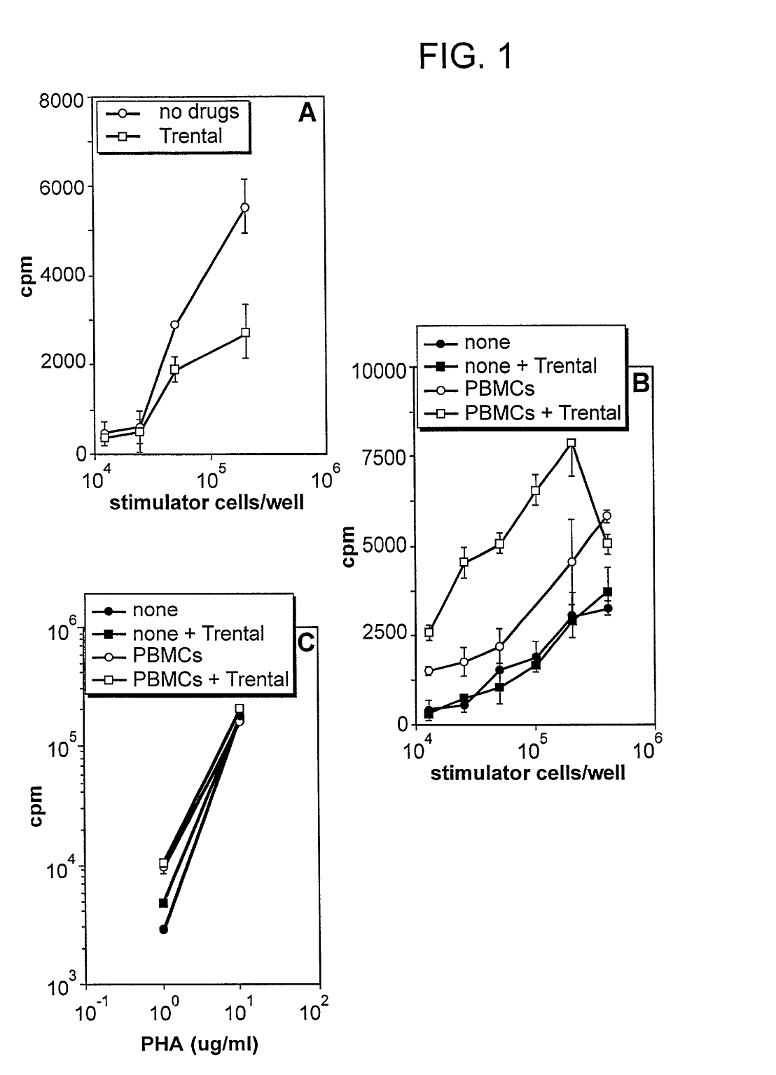

Responder naive T cells can mount a proliferative response against stimulator PBMCs as shown in FIG. 1A. When T cells are primed in vitro and recalled with irradiated PBMCs from the same donor to analyze the memory response, priming of cells resulting in enhancement of proliferative response is apparent as shown in FIG. 1B. Analysis of the effects of POX on both the primary and the memory responses shows that the primary T cell response declines in the presence of POX (FIG. 1A). However, if priming is done in presence of POX, the memory proliferative response is much improved despite the fact that the primary response is inhibited by the drug (FIG. 1B).

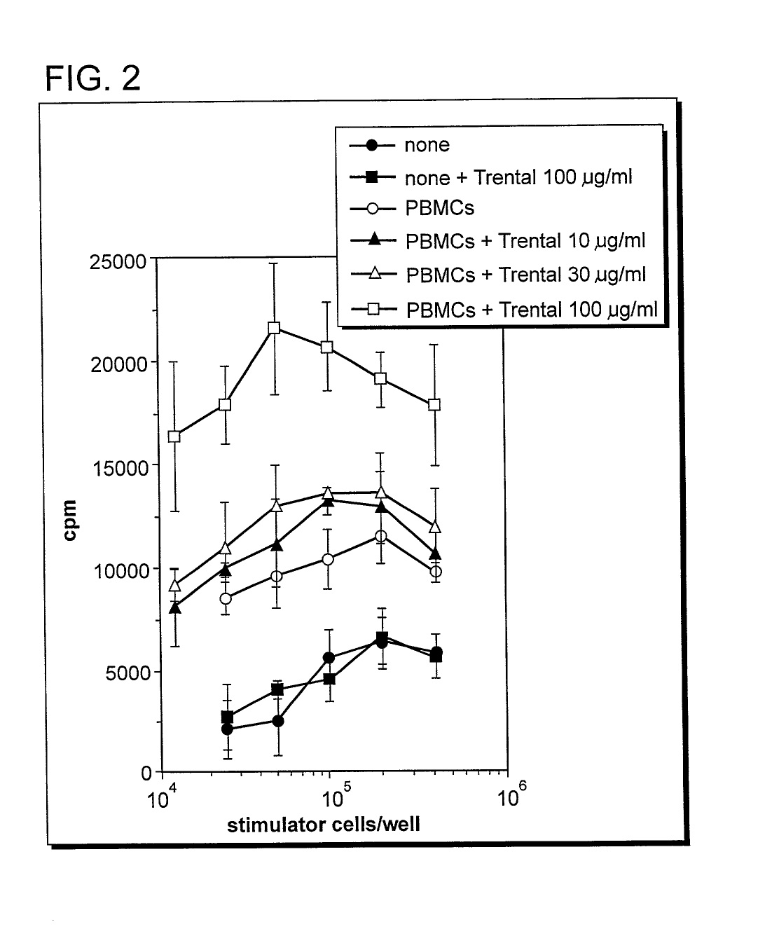

The enhancement of the memory response observed is antigen-specific and is seen only if both POX and stimulators are present during priming, since simply incubating responder cells with POX does not enhance their response (FIG. 1b), and since a polyclonal stimulator of T cells—phytohaemagglutinin (PHA—does not bring about any enhancement of response over and above that seen in the absence of the drug (FIG. 1C). The effect of pantoxifylline can be observed over a wide range of concentrations as shown in FIG. 2.

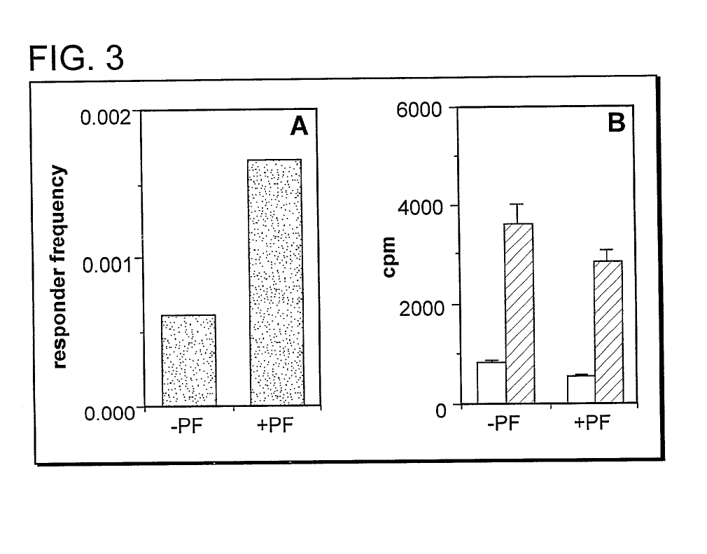

Limiting dilution analyses were used to calculate the number of transplantation antigen-specific memory T cells in cultures primed in the presence or absence of POX. Multiple replicate wells of titrated numbers of primed responders were restimulated. the numbers of wells showing proliferative responses at each responder dose were used to calculate the frequency of responding memory t cells in the primed populations. estimates of the extent of proliferation in wells effectively containing single responder cells served as a measure of clonal burst size. These studies showed that the presence of POX during priming enhanced the frequence of responding memory t cells without altering the clonal burst size of the responders (FIG. 3A and 3B).

Thus, POX differentially affects ‘first’ proliferative responses and the priming process for enhanced memory proliferative responses. Specifically, POX inhibits the first proliferative response, but its presence during priming enhances commitment to immune memory.

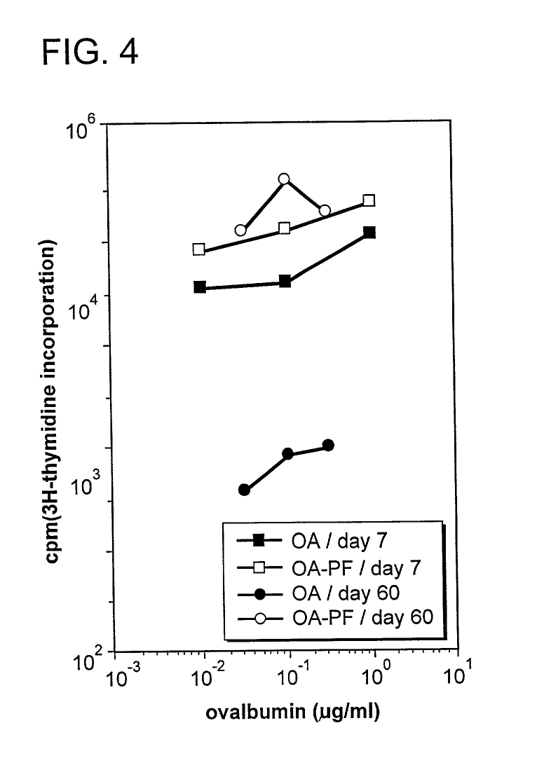

In the second system used to validate the memory-enhancing effects of POX, we have used immunization with a very poor immunogen, egg albumin without adjuvant. It is known that the generation of immune responses is very poor in the absence of adjuvant. The memory T cell responses read out from such immunized mice on day 7 decline to near non-existence by 60 days post-immunization (FIG. 4). In contrast, if the mice are treated with POX during the first five days after immunization, the T cell memory recalled at day 7 is higher to begin with, and stays dramatically high even when it is recalled at day 60 post-immunization (FIG. 4). Thus, POX enhances antigen-specific T cell memory and its duration if used during the first few days after immunization.

These data clearly establish that POX usage in vivo during immunization can enhance the magnitude and the persistence of immune memory. The dose of POX used in these experiments in mice is well within the dose range used for chronic treatment of human patients.

Therefore, we believe POX is entirely usable clinically as an adjuvant for specific enhancement of immune memory during vaccination.

### EXAMPLE 1

We have studied the effects of pharmacological agents present during T cell priming in vitro on the subsequent secondary ‘recall’ proliferative responses mounted by these primed cells. We have shown earlier that the presence of pentoxifylline (POX) during priming of human T cells in vitro by HLA-mismatched PBMCs results in a decrease in primary proliferative responses but enhanced secondary proliferative responses. POX is a drug used extensively clinically for a variety of inflammatory and vascular disorders. It is aphosphodiesterase inhibitor and can induce increases in intracellular cAMP levels. Its effects have been studied on a wide variety of cell types including T cells, B cells and cells of myeloid lineage. In T cells, it has been shown that the effects may be mediated via modulation of induction of transcriptionally active proteins of the rel family. Here we explore the mechanism of action of POX responsible for the enhancement of secondary or ‘memory’ recall responses. We find that the effect of POX is mimicked by enhancement of cAMP levels in T cells, is independent of IL-2, and is associated with alterations in the degree of apoptosis in the responding T cells.

Materials and Methods

T Cell Priming and Proliferation Assays

PBMCs from HLA-mismatched individuals were separated using density-gradient centrifugation on Ficoll-Paque (Pharmacia, Uppsala, Sweden). Stimulator PBMCs were used in titrated doses after g-irradiation (200 rads). For the primary proliferative alloresponses, stimulator in graded doses were used with 1-3×10⁵ responders per well in 200 μl of RPMI-1640 (Life Technologies, Grand Island, N.Y.) fortified with L-glutamine (Life Technologies) and antibiotics, containing 10% heat-inactivated responder autologous serum. Cultures were incubated in a CO₂ atmosphere for five days, pulsed with 0.5 uCi of (H³)-thymidine (New England Nuclear, Boston, Mass.) for the last 8-12 hours of the assay and harvested onto glass fiber filters for counting cell-incorporated thymidine (Betaplate; Wallac, Finland). results are expressed as mean cpm±SE for triplicate cultures.

For priming, responder and stimulator cells mixed in 2-5 ml cultures in a ratio of 1:1-3:1. After five days of incubation, the cells were harvested, counted and viable cells used as primed responders with g-irradiated allostimulators in secondary alloproliferation assays, which were pulsed with (H³)-thymidine after four days for estimating cell proliferation. For flow cytometric analysis, cells were separated from debris by Ficoll-Paque gradient centrifugation before staining.

Dibutyryl cyclic AMP (dbcAMP), pentoxifylline (POX), aphidicolin (all from Sigma Chemical Co., St Louis, Mo.) and recombinant IL-2 (Boehringer Manheim Corp., Manheim Germany) were added singly or together to the culture in various doses as indicated during priming and in primary alloresponse assays. Where fixed PBMCs were to be used as stimulators, they were fixed with 0.05% paraformaldehyde (PF; Sigma Chemical Co.) at room temperature for 20 minutes, washed extensively and used.

Clonal Responder Frequency and Burst Size Analysis

To determine the frequence of all-specific precursor cells in the various responder populations, titrating numbers (from 10,000 to 200 per well) of variously primed cells were added to a constant number (1×10⁵ per well) of g-irradiated stimulator PBMCs. Of the 48 wells plated per responder dose, those wells showing a response at least twice that of the background value were scored positive. The responder frequency was calculated by taking the number of negative wells into account. The response was considered clonal at the responder cell number where the proportion of positively responding wells was less than 37%. At responder cell concentrations showing clonal frequencies, the average cpm values of responding (versus non-responding) wells were used as an estimate of the average clonal burst size of single responding T cells.

Flow Cytometry

For analysis of cell surface markers, primed cell populations separated on Ficoll-Paque gradients were used. All cells used for staining showed >95% viability by trypan blue exclusion. Briefly, 1-3×10⁵ cells per well were stained with primary antibodies at 4° C., followed by appropriately labeled secondary reagents if required. Samples were analyzed using a Bryte flow cytometer (bio-Rad, Hemel Hampstead, UK). Antibodies to the T cell markers CD3 (OKT3), CD4 (OKT4), and to the activation markers CD25 and Fas (CD95) were used for staining. To detect apoptosis using propidium iodide (PI, Sigma Chemical Co.), antibody-stained cells were fixed and permeabilized with 70% ethanol, washed, and PI (10 mg/mil) added just before cytometric analysis. Data were analyzed using FlowJo software (Treestar, San Jose, Calif.).

Results

Enhanced T Cell Priming Seen in the Presence of POX is Due to a Direct Effect of the Drug on Responder T Cells.

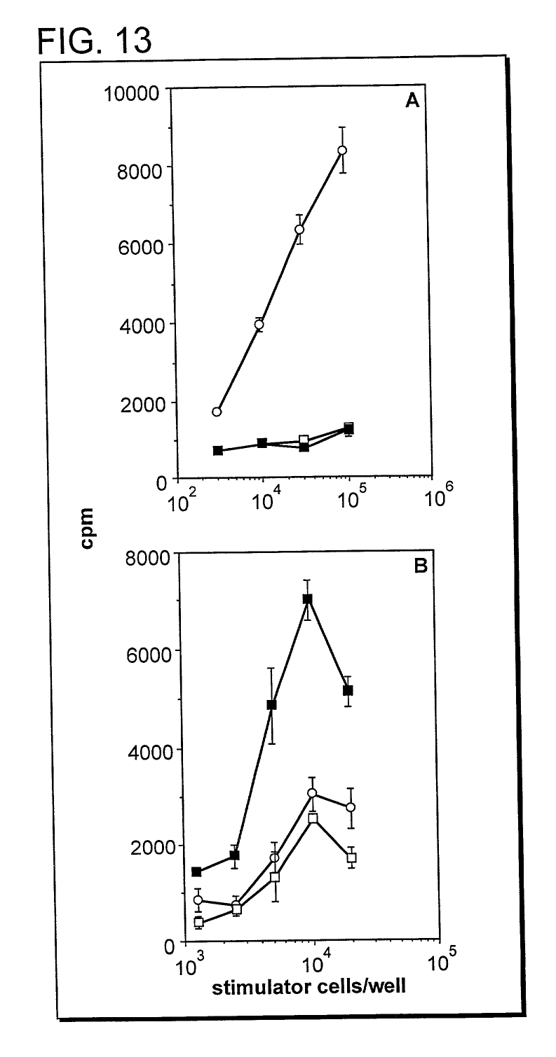

We have shown previously that the presence of POX dur4ing a primary alloresponse in vitro results in dose-dependent inhibition of the immediate proliferative response and preparing the responder T cells for an enhanced secondary response. POX has a variety of effects on the priming APCs, priming was done using PBMCs fixed with 0.05% PF. FIG. 13A shows that the primary response to PF-fixed PBMCs was much lower than that observed for unfixed PBMCs, and was not enhanced by the presence of POX. Fixed PBMCs are not effective at priming T cells for a secondary response either (FIG. 13B). However, T cells primed with such PF-fixed PBMCs in the presence of POX show an enhanced secondary response to unfixed PBMCs (FIG. 13B), suggesting that the effects of POX are mediated directly on responder T cells themselves.

Enhancement in Secondary Alloresponse by dbcAMP

POX is a phosphodiesterase inhibitor and this an enhancer of intracellular cAMP levels (26). It also has a variety of effects on tissue-specific transcription factor induction (27, 28) and is known to downmodulate IL-2 transcription in T cells (21, 29), although it is not clear whether these effects are mediated through increased cAMP levels. In order to begin characterizing the mechanism of action of POX responsible for the enhancement of secondary T cell responses, we tested the effects of a synthetic analogue of cAMP, dbcAMP, which degrades slowly in comparison to cAMP, thereby providing better bioavailability.

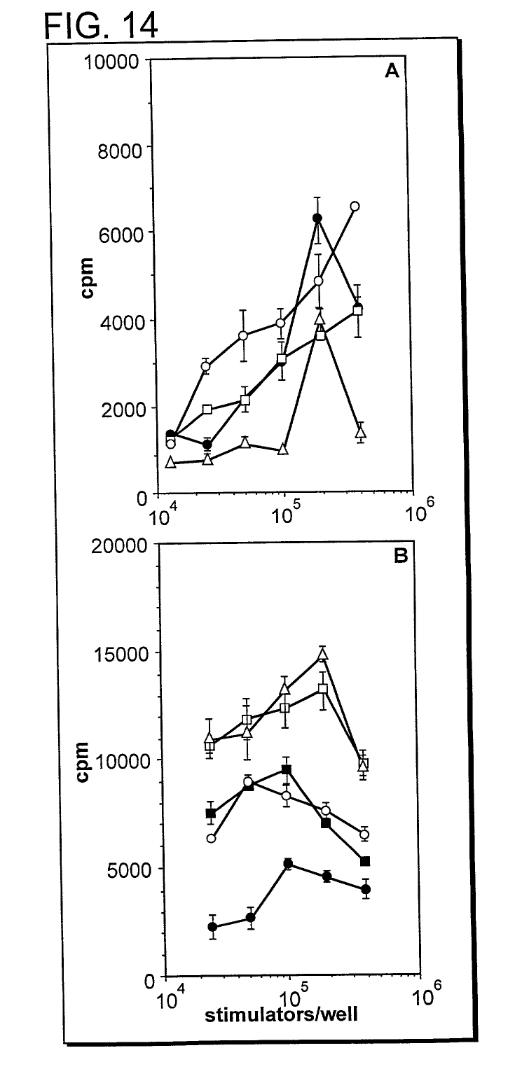

FIG. 14A shows that the presence of 1 mM dbcAMP during primary alloantigen exosure of T cells in vitro inhibits the primary proliferative response. FIG. 14B shows that there is a significant enhancement of the secondary response when priming is done in presence of 100 mM dbcAMP, which does inhibit the primary response. No effect on either primary or recall responses is seen with 10 mM dbcAMP. Thus, high levels of intracellular cAMP during T cell priming is sufficient to diminish the primary proliferative response and enhance the secondary response capability. Presence of IL-2 during priming does not affect enhancement in priming mediated by POX.

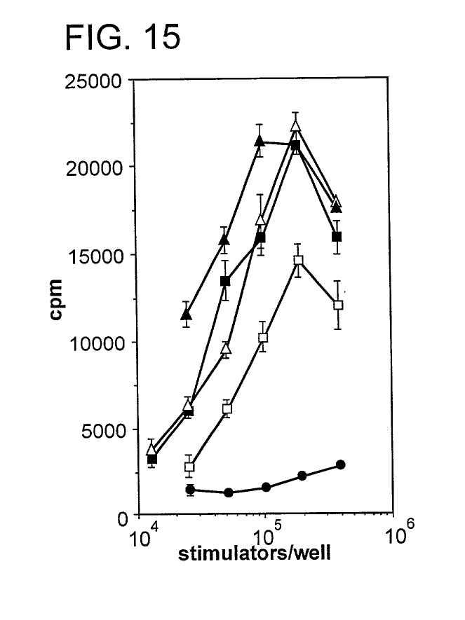

POX is known to suppress transcription of IL-2. To examine whether this lack of IL-2 could account for the enhanced commitment to secondary responses we observe, exogenous IL-2 (3 U/ml) was added during priming in the presence or absence of POX. responder cells primed in the presence of both POX and IL-2 exhibit an enhancement of the secondary response similar to that caused by the presence of POX alone during priming (FIG. 15). Addition of higher or lower concentrations of IL-2 (1-10 U/ml) during priming showed similar results. These data suggest that the suppression of IL-2 transcription is not responsible for the augmentation of the secondary response by the presence of POX during priming. The enhancement of the secondary response by POX is due to an increase in the clonal frequency of proliferation-competent responders.

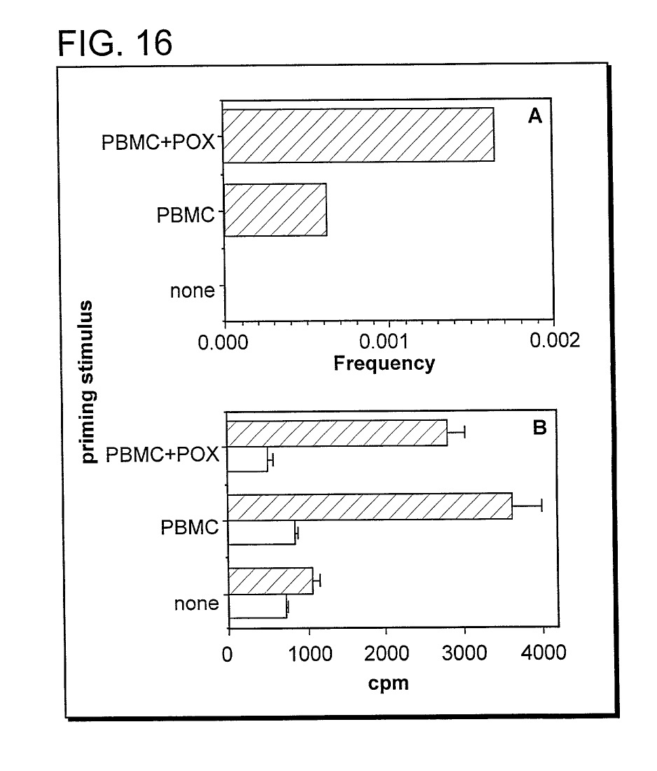

We next estimate the frequency of the alloreactive responder cells after priming by titrating the number of primed cells added per well in the presence of fixed numbers of stimulator PBMCs. FIG. 16A shows that, the PBMC-primed responder population shows a frequency higher than that in unprimed cells, and the presence of POX (or dbcAMP, data not shown) during priming significantly enhances the frequency of responders. This assay also allows the estimation of the ability of a single allospecific cell to give rise to progeny. the average magnitude of the proliferative response in responding wells at clonal frequency (positive wells <37%) was considered a reflection of clonal burst size. As shown in FIG. 16B, the average clonal proliferative response was not significantly different between POX treated and untreated groups, suggesting that differences in the burst size were not contributing to the enhancement in the secondary response observed with POX. Taken together, these data suggest that the clonal frequency of responders is enhanced by priming in the presence of POX. CD25 is an early activation marker associated with alloprimed cells.

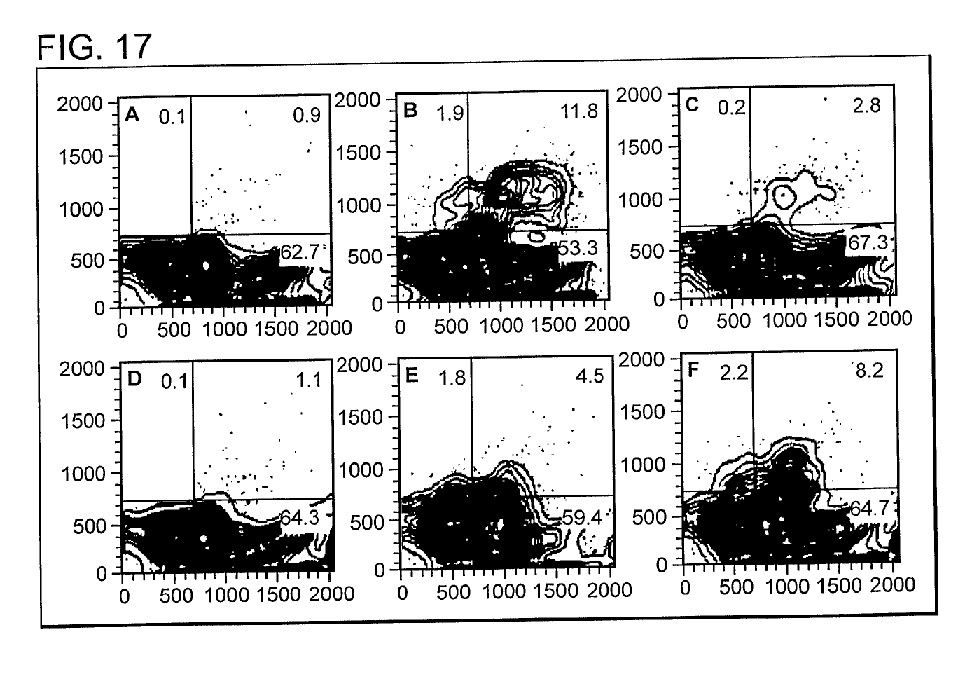

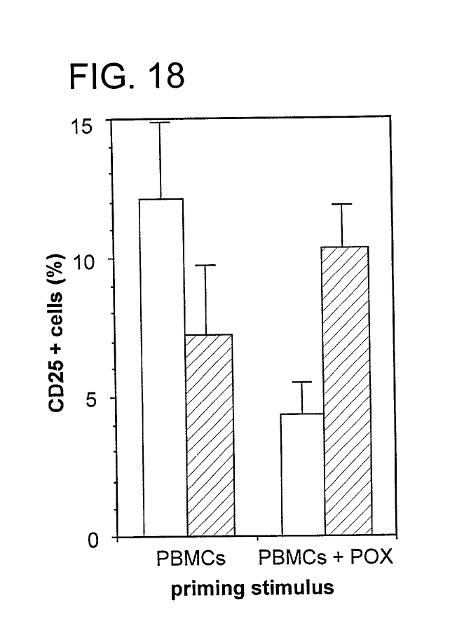

The phenotype of CD4 T cells activated during priming in vitro was examined next using various markers associated with T cell activation. Antibodies against CD25 (IL-2Ra), CD26, CD45RO, CD69 and gp240 were used, but except for CD25, none of the other markers gave consistent and reliable results (data not shown). CD25 expression on CD4 T cells in the responder populations was examined in a two-color flow cytometric analysis at two separate time points,—at the end of six days of priming and after two further rest days in culture after washing and resuspension in resh medium without any stimuli. Most CD25-expressing cells in the primed cultures were CD4 T cells. Only a very small proportion of CD4 T cells showed CD25 expression in the unprimed population (FIGS. 17A and 17D). The frequency of CD25 expression increased in the PBMC-primed responders (FIG. 17B) and this percentage was lower if POX (100 μg/ml) was present during priming (FIG. 17C). However, this pattern altered during two days of rest in culture, and while the frequency and level of CD25 expression went down in the population primed without any drugs (FIG. 17E), cells primed in the presence of POX showed increased frequency of CD25 expression although with some decrease in the level of expression (FIG. 17f). Similar data were observed if dbcAMP was present during priming (data not shown). Data from many such independent experiments are summarized in FIG. 18. It can be seen that the proportion of CD25* cells was reproducibly lower if POX was present during priming with allo-PBMCs. However, after 48 hours of rest in culture, the frequency of CD25* cells declined in the populations primed in the absence of POX, while it went up in the group primed under POX cover.

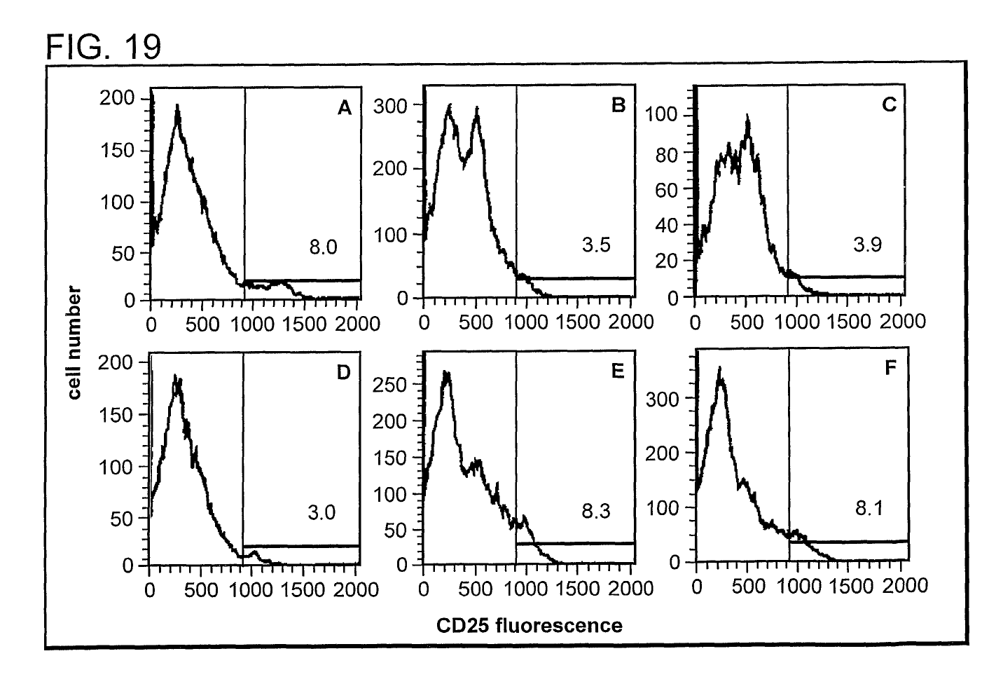

This relative increase in the CD25+ CD4 T cells in the POX-treated group during 48 hours of rest could be due to one of the two possibilities. One is that the withdrawal of POX from the culture during the rest period results in depression of proliferation of allospecific T cells, while the other is that T cells alloprimed under POX cover are surviving far better in culture compared not only to unprimed bystander T cells but also to T cells primed in the absence of POX. In order to examine these possibilities, we added aphidicolin, a DNA polymerase inhibitor, to inhibit cell proliferation during the rest period. Aphidicolin was titrated for inhibition of primary alloproliferative responses (data not shown), and based on that titration, a dose of 1 mg/ml of aphidicolin was used to inhibit proliferation during the two day rest period. FIG. 19 shows a flow cytometric analysis of such an experiment, in which the frequence of CD25* cells during the rest period without aphidicolin (panels C and F respectively). Thus, the presence of POX during priming induces higher levels of CD25 with slower kinetics of downmodulation.

Delayed Apoptosis of Activated T Cells May Contribute to the Augmentation of Secondary Response by POX.

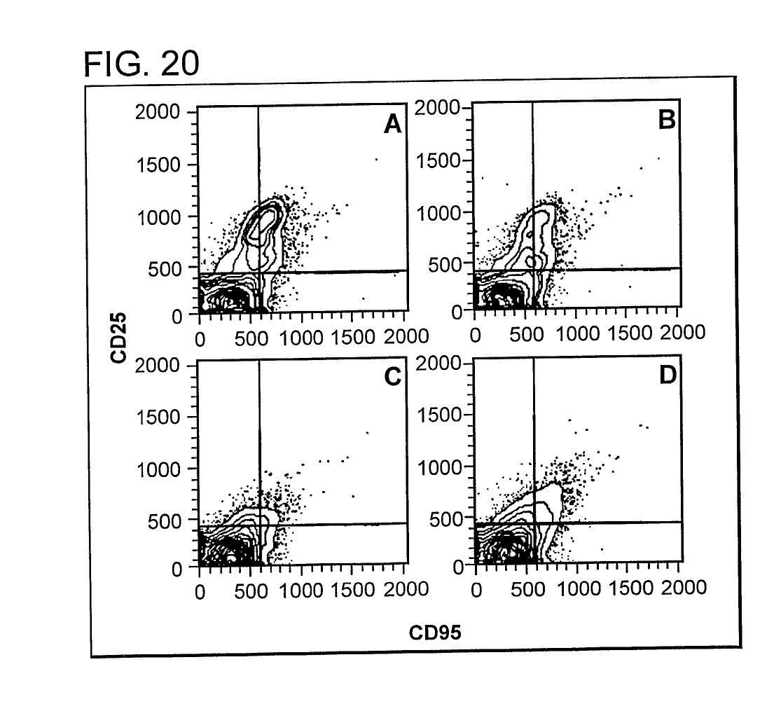

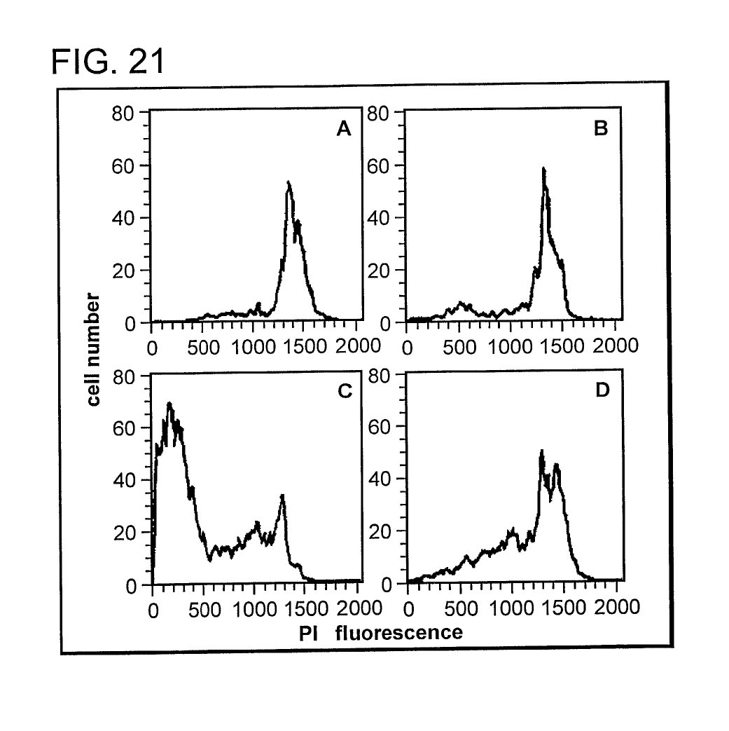

The differences between the kinetics of CD25 expression over two days of rest culture prompted the question of whether this was linked to effects of POX on the survival of T cells activated during priming. Almost all CD25⁺ cells generated during priming were CD4 T cells (FIG. 17A). therefore, responder populations primed variously were stained in a two-color flow cytometric analysis for CD25 and Fas, since Fas engagement is involved in activation induced T cell death. The frequency and level of Fas expression in the CD25⁺ population was unchanged in the presence (FIG. 20, panels B and D) or absence (panels A and C) of POX during priming. The frequency went down in both the groups at the end of two day rest period (panels A and B—day 0, panels C and D—day 2). On the other hand, two-color flow cytometric analysis for CD25 and PI for apoptosis showed that the bilk of the T cells activated during priming die by apoptosis during the subsequent 48 hours (FIG. 21, panels A and C), so that PI staining shows a greatly reduced DNA content in them. However, if priming was done in the presence3 of POX, the proportion of apoptotic cells in the CD25⁺ population does not increase significantly over the 48 hour rest period (panels B and D). Thus, POX clearly inhibits apoptosis in primed T cells.

It must be noted that the cells being analyzed here were all viable cells isolated on Ficoll-Hypaque gradients and excluding trypad blue efficiently. The viable cell numbers recovered form these cultures were consistent with the inhibition of primary proliferative responses by POX. thus, unprimed cultures yielded 0.25±0.07, PBMC-primed populations had 0.38±0.05, while cultures primed under POX cover gave 0.27±0.06 million viable cells per million input cells. Clearly, the presence of POX did not cause any non-specific effects on cell viability in these cultures.

Discussion

T cell activation eventually leads to the generation of two separate types of functionally differing progeny populations;—effector and memory. Effector cells may be end-stage T cells as are plasma cells in the B cell lineage, and if still alive, they secrete cytokines or deliver cytotoxic signals immediately upon antigenic re-exposure rather than undergoing proliferation. In contrast, memory cells give rise to more memory cells as well as to fresh effector progeny upon reactivation. While the immediate consequences of T cell activation have been extensively analyzed, signaling pathways regulating such physiological and more long-term results of activation are less well understood. Part of the problem is the difficulty of phenotypic distinction between effector and memory T cells in the experienced T cell compartment, which does have some phenotypic markers such as CD44 cells, although the stability of even that distinction is uncertain and controversial. In the absence of clear phenotypic identification of effector and memory T cells, indirect methods are resorted to in trying to dissect the factors and mechanisms involved in regulating the balance between effector and memory T cell commitment.

In this context, we have been trying to analyze factors contributing to differential commitment of T cells to immediate responses versus secondary proliferative response capabilities. We have shown previously, in a system of T cell priming in vitro using allorecognition of MHC-mismatched human PBMCs, that T cell priming for a recall proliferative response does take place even if the immediate proliferative response is blocked using the DNA polymerase inhibitor aphidicolin, and that while the phosphodiesterase inhibitor POX inhibits the immediate proliferative response, its presence during priming enhances t cell recall response capabilities (18). We have now further explored the mechanisms by which POX mediates these effects on T cell priming.

The first issue is whether the immunomodulation by POX that we observe is mediated by its effects on T cells directly, or whether it works indirectly by altering the APC functions of the allostimulator PBMCs used for priming. PF fixation of stimulator APCs leads to poor primary proliferative responses as well as to poor priming for secondary responses. POX does not affect the primary response to such PF-fixed metabolically inactive stimulators, but its presence during priming enhances the subsequent secondary response to unfixed stimulator APCs (FIG. 13). Thus, the direct6 effects of POX on T cells is sufficient to induce the effects on priming we observe, and makes the alterations in signal transduction pathways brought about by POX in T cells 919, 23, 26) directly relevant.

Several effects of POX on T cell signaling pathways have been reported. Although POX is a phosphodiesterase inhibitor, it does not necessarily follow that all its effects are a consequence of that property. A major consequence of inhibition of phosphodiesterase is an increase in the levels of intracellular cAMP. The data in FIG. 14 suggest, prima facie, that the elevation of cAMP levels brought about by POX is likely to be sufficient to mediate the effects of POX on T cell priming, since the long-lived cAMP exerts similar effects. However, we observe in preliminary experiments that POX does not increase the intracellular cAMP levels in T cell lines significantly, unlike dbcAMP (data not shown). This raises the possibility that although dbcAMP mimics POX in its effects on T cell priming, the precise molecular pathways involved may differ.

Elevation of cAMP levels inhibits IL-2 production by activated T cells, and POX itself is a potent inhibitor of IL-2 transcription. There are reports that blocking of IL-2 functioning by the addition of anti-IL-2 receptor antibodies during T cell priming may enhance secondary response-enhancing effect of POX is mediated through its inhibition of IL-2 transcription. If this were the case, the effect would be expected to be blocked by the addition of exogenous IL-2 in the priming cultures. Since POX still enhances T cell priming despite the presence of IL-2 (FIG. 15), it is evident that the critical molecular target/s of POX/cAMP involved in enhancing T cell commitment to secondary responsiveness is not likely to be IL-2.

It can be seen from FIG. 13 and previous data that the allospecific T cells proliferate more extensively during priming in the absence of cAMP/POX. Thus, the numbers of antigen-specific T cells would be expected to be greater at the end of the six-day period of priming in cultures primed in the absence of dbcAMP of POX. Yet, paradoxically, the bulk secondary response is greater if levels of cAMP are higher in the priming cultures. There are a number of non-exclusive possibilities to explain this. One possibility is that, although there is indeed a greater number of antigen-specific T cells in the cultures primed without POX, a greater proportion of them are end-stage effectors and cannot proliferate again in response to a secondary stimulus. A second possibility is that POX inhibits activation-induced T cell apoptosis so that while a large proportion of the responding T cells in cultures primed in the absence of POX die, the proportion of such dying cells is less in cultures in the presence of POX. We have attempted to examine both possibilities.

When limiting-dilution assay-based estimates of the frequence of proliferation-competent allospecific precursors in primed cultures are made, it is seen that priming in the presence of POX increases this frequence, but there is no change in the clonal burst size per precursor (FIG. 16). These data suggest that POX may be simply altering the balance between the commitment of responding T cells to terminal effector cells versus secondary responder cells, rather than qualitatively changing the properties of the secondary T cells generated so as to enable them to mount a larger response per cell. Similar data were obtained with dbcAMP (data not shown).

To examine the issue phenotypically, we have looked at the expression of cell surface molecules that are T cell activation markers. While many markers used,—CD26, CD45RO, CD69 and gp240,—did not give any consistent results in independent experiments, there are interesting alterations caused by POX in CD25 expression patterns. CD25, or IL-2Ra, is expressed on activated T cells and enhances the ability of T cells to respond to IL-2 by increasing the affinity of the low-affinity IL-2rb found on all T cells. Priming in the presence of POX decreases the phenotypic frequency of activated CD25⁻ T cells seen at the end of six days of priming (FIG. 17). Yet, despite this reduction in the number of activated antigen-specific T cells by POX, there is an increase in the frequency of proliferation-competent allospecific T cells (FIGS. 14 and 16). These data suggest that POX increases the frequency of proliferation-competent progeny generated from responding T cells. Again, similar results are seen with dbcAMP (data not shown).

The IL-2R itself is unlikely to be a major factor in the enhancement of secondary responsiveness of T cells primed under POX cover, since the presence of exogenous IL-2 during priming does neither enhance the recall capability on its own, nor does it block the enhancement induced by POX (FIG. 15). This is particularly significant since signaling of activated T cells by IL-2 via IL-2R is critical for activation-induced T cell death. If the role of IL-2 depletion in preventing apoptosis and permitting responder T cell survival was critical for the enhanced secondary responsiveness induced by POX the POX-mediated enhancement should be blocked by the presence of exogenous IL-2. That this does not happen further emphasizes that the effects of POX are unlikely to be mediated through the IL-2/IL-2R pathway.

However, when considering the expression of CD25 as a marker of T cells activated during priming, it can be seen that the rate of loss of CD25⁺ T cells during two days of rest after priming is altered in populations primed under POX cover (FIGS. 17 and 18). In responder cell populations primed without any POX, the frequency of CD25⁺ cells falls rapidly over two days of rest. However, if priming is done under POX cover, the frequency of the CD25+ cells rises significantly over the two-day rest period, so that the addition of aphidicolin during rest is needed (FIG. 19) to insure that this is a consequence of increased survival rather than proliferation. It is also possible that a decline in the levels of CD25 on primed cells over the 48 hour rest period leads to the apparent loss of CD25⁺ cells in the responders primed without POX cover. However, if this was a major factor in the CD25⁺ cells in responders primed under POX cover would be expected to be correlated with sustained levels of CD25 expression on them, which does not appear to be the case (FIG. 17). Therefore, it was plausible to examine the possibility that POX mediates enhanced survival of primed T cells.

If such differential survival of responding T cells postpriming is involved in the enhancement of secondary responsiveness brought about by POX, it is most likely to be mediated by inhibition of apopotosis, since induction of the apoptotic pathway is a major outcome of T cell activation. Apoptosis of activated T cells involves Fas (CD95)-mediated signals. When the levels of Fas are examined on CD25⁺ activated T cells in culture at the end of six days of priming, it can be seen that cells primed under POX cover show frequencies and levels of Fas expression similar to cells primed in the absence of POX (FIG. 20), suggesting that the induction of Fas expression during T cell priming is unaffected by POX. However, the effective induction of apoptosis is inhibited nonetheless, as shown by the high frequency of CD25+ cells staining poorly with PI (FIG. 21). We have noted similar effects with dbcAMP as well as (data not shown). This suggests that death pathways downstream of Fas may be affected by POX.

Thus, using in vitro priming of T cells as a model system, we show that the enhancement of T cell effect that can also be mimicked by enhancement is accompanied by reduction in the activation-induced apoptosis of primed T cells.

### EXAMPLE 2

Isolation Of Cell Populations

Responder and stimulator PBMCs were separated from heparinized blood by centrifugation on Ficoll Paque (Pharmacia, Uppsala, Sweden) density cushions. Cells were cultured in RPMI 1640 medium (Life Technologies. Grand Island, N.Y.) containing L-glutamine (Life Technologies) and antibiotics (Hi-Media, Bombay, India) with 10% heat inactivated (56° C. for 30 min) responder-analogous serum at 37° C. in 5% CO₂. Stimulator PBMCs were γ-irradiated at 2000 rad using a CO⁶⁰ source (Bhabha Atomic Research Centre, Bombay, India) before use.

Fixation of Stimulator PBMCs by Paraformaldehyde

Wherever appropriate, stimulator PBMCs were fixed with various concentrations of paraformaldehyde (PFA, Sigma Chemical Co., St. Louis, Mo.) after γ-irradiation. The PBMCs were incubated in 1 ml of serum-free RPMI 1640 containing the appropriate concentration of PFA for 20 minutes at 37° C. in a 5% CO₂ atmosphere. After incubation, the cells were washed extensively and were re-suspended in medium containing 10% responder-autologous serum before use.

T Cell Priming and Proliferation Assays

PBMCs from MHC-mismatched donors were used as responder-stimulator pairs. Stimulator cells were used after γ-irradiation (200 rad) at various concentrations, as shown. Proliferation assays were set up as parallel cultures and were pulsed and harvested as below on different days when necessary for the analyses of the kinetics of the response. Where appropriate, cyclosporin A (CsA, Sandoz, Milan, Italy), aphidicolin (Aph, Sigma Chemical Co., and/or pentoxifylline (Trental, Sigma Chemical Co.) Were added at the concentrations indicated, or PFA-fixed PBMCs were used. The plated were pulsed with 0.5 to 1 μCi of (³H)thymidine ([³H]TdR. Amersham, Little Chalfont, U.K.) During the last 12 to 16 h of the assay (4 days for a recall assay and 6 days for a primary assay, unless indicated otherwise). [³H] TdR incorporation was measured by harvesting the cultures on glass-fibers and counting in a scintillation counter (Betaplate: LKB-Pharmacia. Uppsala, Sweeden). The results are expressed as mean cpm±SE of triplicate cultures.

For priming in vitro, responder PBMCs were incubated for 72 or 144 h at a ratio of either 1:1 or 2:1 respectively, with stimulator PBMCs. Where appropriate, CsA. Aph. and/or Trental were added at the concentrations indicated, of PFA-fixed PBMCs were used instead. Cells were then harvested and washed extensively, and viable cells were counted and used as primed responders in a recall assay with freshly isolated and irradiated stimulator PBMCs or with graded doses of either IL-2 (Boenringer Mannheim Corp., Manaheim, Germany) or PHA (Boehnnger Mannheim Corp.), along with 1×10⁵ irradiated responder PBMCs.

Dimunition in T Cell Proliferation-inducing Ability of PFA-treated PBMCs is not Necessarily Matched by a Similar Loss in Their Priming Potential

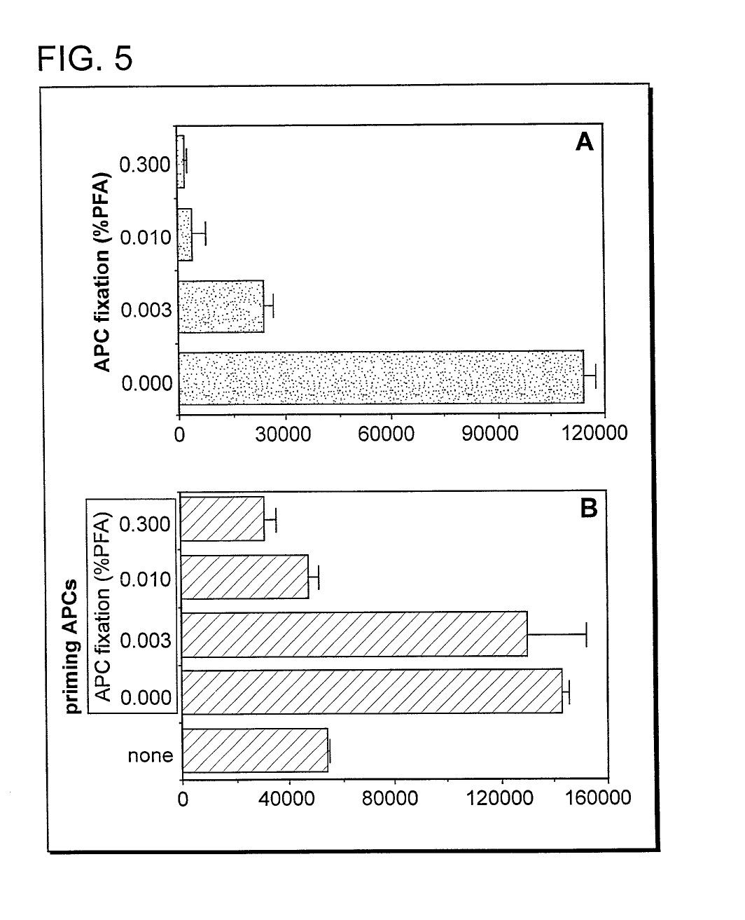

Fixation of APCs with PFA has been shown to decrease their co-stimulatory capacity for T cell activation. We have therefore varied the fixing concentration of PFA required to inhibit the induction of a primary alloproliferative response. With increasing concentration of PFA used for fixation, there is greater decrease in the first response, but even the use of 0.0003% PFA leads to a significant lowering of the response (FIG. 5A). For analyzing the effect of PFA fixation of APCs on the priming of responder T cells for enhancement of a recall response, responder PBMCs were primed with PFA-fixed or unfixed PBMCs at 1:1 ratio for 72 h. These primed cells were then restimulated with fresh γ-irradiated stimulator PBMCs. FIG. 5B shows that, as expected, unfixed APC-primed responder cells show an enhanced recall response as compared with the unprimed control population. Responder cells primed with APCs fixed at high concentrations of PFA (0.3%) show recall responses distinctly lower than those of unprimed cells, suggesting that, in addition to the decrease in the primary proliferative response, there has been some induction of T cell tolerance, as has been demonstrated in other systems. However, APCs fixed at 100-fold lower concentration of PFA (0.003%) induce a recall response capability comparable with or greater than unfixed APCs do (FIG. 5B), despite the lowering of their primary proliferation-inducing potential. This, a decrease in the primary proliferative T cell response is not necessarily accompanied by a decrease in the ability to be primed for an enhanced recall response.

Inhibition of DNA Replication During t Cell Stimulation Still Permits Efficient Priming for a Recall Response

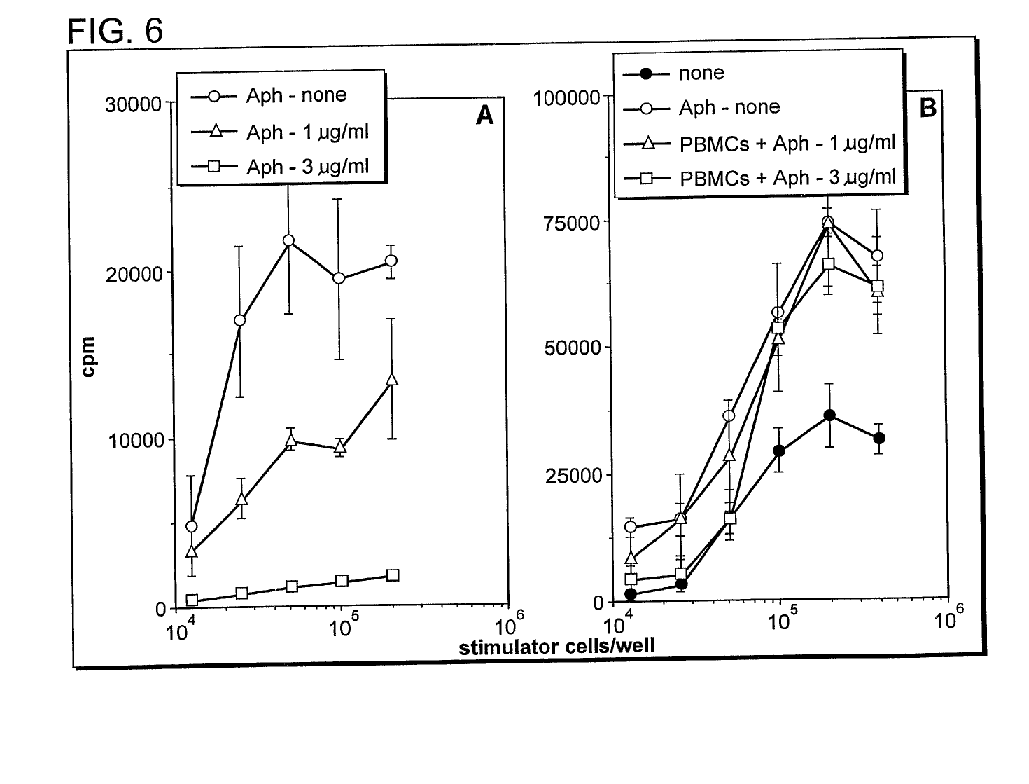

Since a decline in the first proliferative response was not necessarily accompanied by a decline in T cell priming for a recall response, we attempted to analyze, specifically, the effect of cell multiplication itself on priming. Aph is a reversible DNA polymerase inhibitor; and as a result, prevents DNA replication and thymidine incorporation during a primary proliferative T cell alloresponse in a dose-dependent manner, as shown in FIG. 6A. FIG. 6B shows the effect of the presence of Aph during T cell priming on the recall proliferative response to γ-irradiated allo-PBMCs. When T cell proliferation is inhibited substantially using 1 μg/ml of Aph during priming (as shown in FIG. 6A), the recall response of the primed cells is comparable with that of responder cells primed in the absence of Aph. A higher concentration of Aph (3 μg/ml) results in completely undetectable proliferation of T cells in the first response, but even these cells show a significant enhancement of their recall response. The responses or unprimed PBMCs incubated with Aph are unaltered (data not shown). These data indicate that, for T cell priming to take place, clonal expansion of the unprimed cells is not an absolute requirement.

Presence of Trental During Priming Decreases Primary Responses, but Enhances Recall Responses

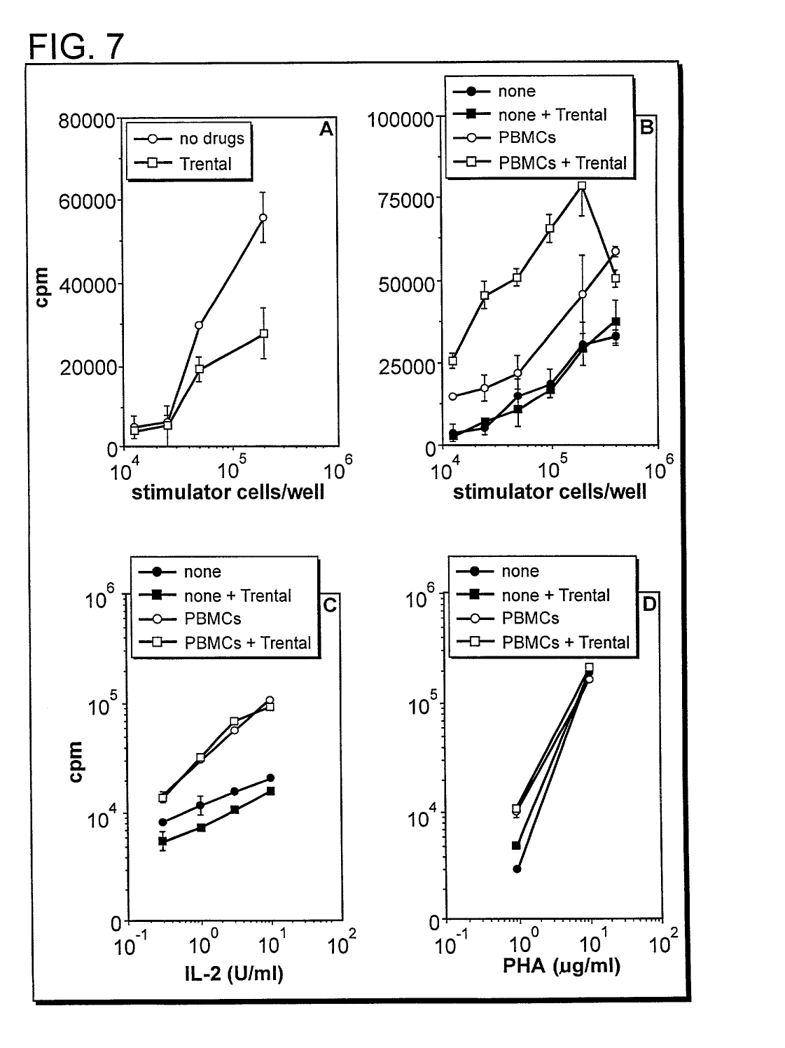

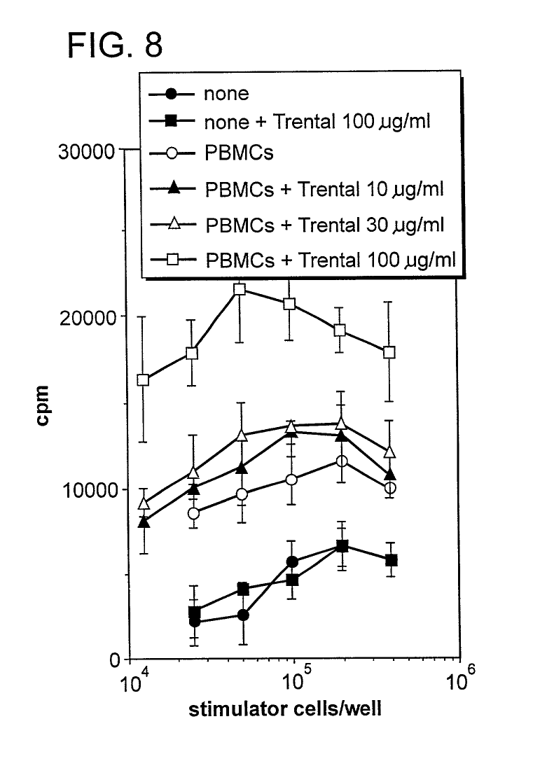

Trental is a phosphodiesterase inhibitor and increases intracellular concentrations of cAMP. It has been reported that it down modulates IL-2R expression and IL-2 synthesis. Since IL-2 is an important growth factor for T cell multiplication, we have examined the effect of Trental on both primary T cell responses and on T cell priming. Preliminary titrations showed that there was a decrease in the primary proliferative response with increasing doses (1-300 μg/ml) of Trental (data not shown), and 100 μg/ml of Trental was selected as the dose to work with. FIG. 7A shows that the primary alloproliferative response is reduced significantly in the presence of 100 μg/ml of Trental. On the other hand, when T cells are primed with this dose of Trental for 6 days and restimulated in the absence of the drug, the recall proliferative response is enhanced over and above that of cells primed with PBMCs in the absence over and above that of cells primed with PBMCs in the absence of the drug (FIG. 7B). Trental does not cause any nonspecific enhancement of recall responses, since incubation of unprimed PBMCs with Trental at 100 μg/ml does not enhance their subsequent alloproliferative response (FIG. 7B). T cells primed in their presence of 100 μg/ml of Trental respond to IL-2 to the same extent as those primed in the absence of the drug IL-2 to the same extent as those primed in the absence of the drug (FIG. 7C), suggesting that the induction of IL-2R may not be affected by Trental. The enhancement of recall responses by Trental is Ag specific, since the response of T cells primed in the presence or absence of the drug the PHA is comparable (FIG. 7D). With lower doses of Trental, the enhancing effect of the recall response decreases, although there is no inhibition below the level achieved in the absence of the drug (FIG. 8). Thus, Trental causes a diminution in the primary response while including an enhancement of the primed state.

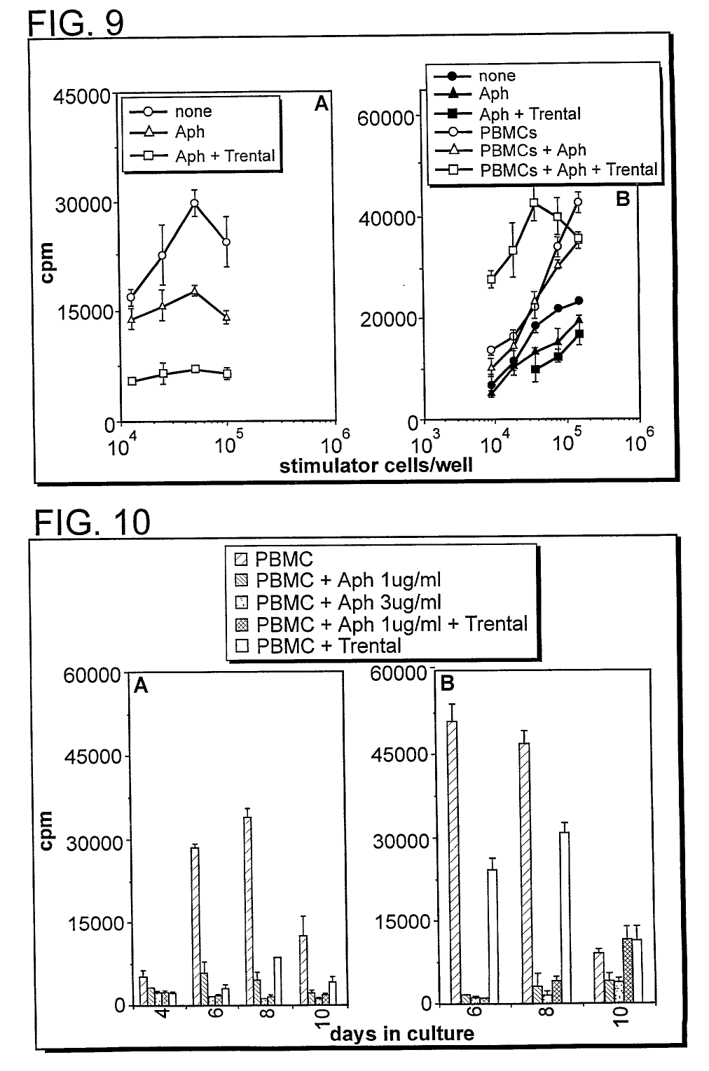

To examine whether Trental can also enhance priming of non-replicating cells, both Trental and Aph were added simultaneously during T cell priming. FIG. 9A shows that the addition of Trental to cells on 1 μg/ml of Aph further decreases the already low primary proliferative response. However, the recall response of T cells thus primed is still higher than that of cells primed in the presence of Aph alone (FIG. 9B). Addition of drugs without priming stimulator PBMCs has no significant effect. Thus, in spite of incorporating an inhibitor of DNA replication during the priming event, the ability of Trental in induce enhanced recall responses remains unaffected.

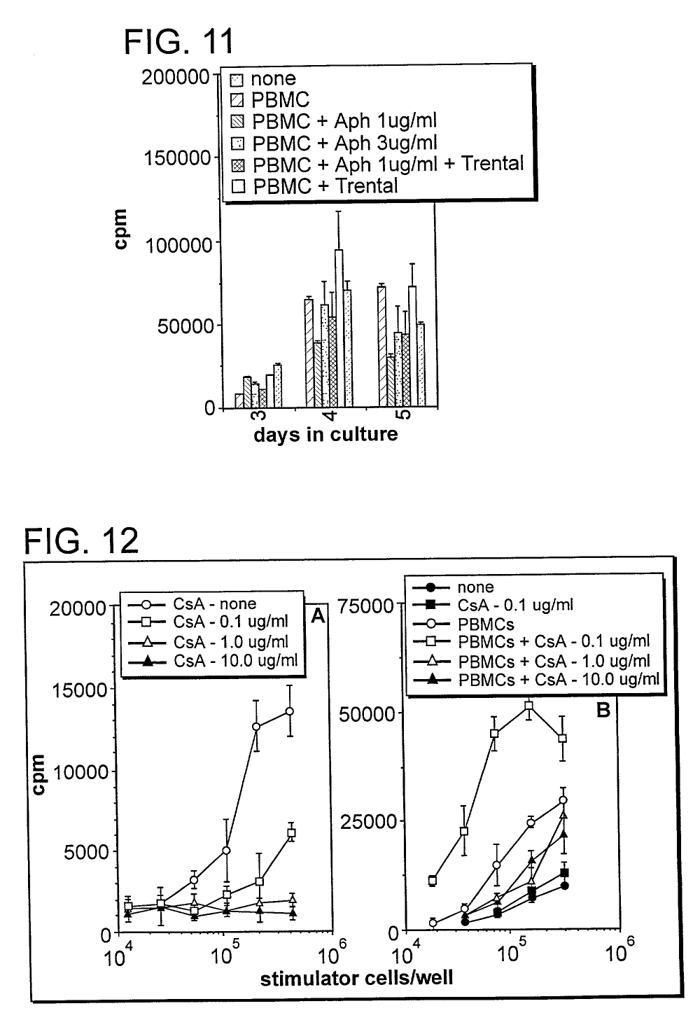

To ensure that the enhancing effects of Trental on the secondary proliferative responses were not simply a matter of delayed primary responses, the effects of the drugs on the kinetics of the primary response were also examined. The primary response to allo-PBMCs was measured at days 4, 6, 8 and 10 in the presence or absence of 1 or 3 μg/ml Aph. 100 ug/ml Trental, or a combination of 1 μg/ml Aph and 100 μg/ml Trental. FIG. 10A shows that the response goes up between days 4 and 6 and declines equivalently by day 10, despite the presence of drugs. However, it still remained possible that the response seen on day 4 of recall stimulation was simply a recovering primary response after drugs had been removed from the medium, To examine this, primary responses were set up as in FIG. 10A, but drugs were removed on day 6 by washing the cells and cultures continued for pulsing on days 8 and 10. FIG. 10B shows that, even in the absence of drugs between days 6 and 10, there is no increase in the primary response in cultures treated with Trental and/or Aph, since all primary responses decline equivalently by day 10. Thus the enhancement of recall responses seen under the influence of Trental is manifested only upon restimulation with allo-PBMCs. And therefore appears to be a true enhancement of secondary or memory responses. The kinetics of the secondary response was also analyzed to investigate the possible basis of the enhancement brought about by Trental. FIG. 11 shows that, while the primary response showed a gradual increase from days 3 to 5, secondary responses peaked at day 4 and began declining on day 5, irrespective of the drug combinations used during priming.

Presence of Low Doses of CsA During T Cell Priming Results in an Enhanced Ability for Recall Responses

CsA is used extensively in clinical practice as an immunosuppressant. It is known to inhibit the transcriptional activation of cytokine genes, notably IL-2, in a calcium dependent fashion and it suppresses T cell proliferative responses. We therefore compared the effects of CsA on primary proliferative responses vs. on T cell priming. FIG. 12A shows that with increasing doses of CsA, the primary alloproliferative response decreases as expected. However, when responder cells primed with allo-PBMCs in the presence of low concentrations of CsA (0.1 μg/ml) are challenged with the same allo-PBMCs, the recall response mounted by these responders is enhanced over that of responders primed in the absence of the drug (FIG. 12B) as the concentration of CsA increases, this enhancement of priming is lost, unlike the effect of Trental, in which, with increasing drug concentration, the enhancement of priming increases. Incubation with CsA alone has no priming effect (FIG. 12B and data not shown).

Discussion

T cell activation has two clearly separable useful consequences. First, as a consequence of well-studied signal transduction and transcriptional activation cascades. There are proliferation and secretion of various cytokines through which effector functions are manifested. Second, there is also a long-term effect of at least some of the responding T cells of their progeny, such that they are converted into memory T cells capable of proliferating more easily and better in response to antigenic re-exposure, and of producing modified cytokine profiles. Thus, any responding T cell population must fulfill these two opposing destinies: creating both active effector T cells producing various cytokines and interacting with B cells or macrophages of other target cells as the case may be, and quiescent memory cells that would respond only upon antigenic re-exposure.

We have been investigating questions related to the generation of T cell memory with an experimental system of T cell activation through allorecognition, in which both a first T cell response as well as an enhanced recall response can be measured in terms of proliferative capabilities, and in which the induction of priming or conferring of the ability to respond better in a secondary response can be conducted in vitro.

T cell activation requires costimulatory signals in addition to the Ag-specific MHC-peptide complex. The B7 family of molecules has been identified as a major contributor to costimulatory interactions, but less well-characterized B7-independent costimulatory pathways also appear to exist. Costimulatory signals appear to be fixation sensitive, and this fixation of APCs leads to a loss in the ability of the APCs to generate a primary T cell so stimulated to respond to completely competent stimuli later, a phenomenon of T cell tolerance termed anergy. Thus, both the effector and the memory responses of a T cell are abrogated by Ag presentation on fixed APCs. We have therefore attempted to examine whether the effects of APC fixation on these two responses are well correlated by titrating the concentration of the fixation agent. PFA as a fixative concentration decreases the ability of the APCs to induce the first proliferative response increases, but even at a low fixative level of 0.003% proliferative responses induced are below those induced by unfixed APCs (FIG. 5A). The recall proliferative responses however, show a different trend. At high fixative concentrations, as has been shown in other systems, the APCs induce T cell tolerance so that the recall response is now less than that of unprimed T cells that have never seen Ag at all (FIG. 5B). But at low fixative concentrations, not only do the APCs not induce tolerance but they induce priming to levels comparable with those achieved by unfixed APCs despite not being able to induce a comparable primary response. Thus, it is possible that costimulatory requirements for the induction of a primary response may be more exacting than those needed for the induction of immune memory.

Curiously, a decrease in the primary proliferative response did not necessarily lead to a decrease in the recall response in these experiments, implying that clonal expansion during first exposure was not necessarily related to the strength of the recall response. To investigate this issue further, we have used the incorporation of a DNA polymerase inhibitor, Aph during the priming step. The effects of Aph are reversible so that cells primed in its presence should be able to proliferate normally once the drug is removed. FIG. 6 clearly shows that inhibition of proliferation in the primary response under the influence of Asph has very little effect on the enhancement brought about in the recall response. These data indicate that clonal expansion is not the major factor responsible for the enhanced responsiveness that is characteristic of T cell memory. It is far more likely, thus, that the increased ability of individual primed T cells to be stimulated is the basis of the enhanced responses characteristic of immune memory.

In fact, in both sets of experiments to date, either APC fixation of Aph results in a decrease of the primary proliferative response or Aph results in a decrease of the primary proliferative response. Thus, Ag-specific T cell numbers in populations primed with fixed APCs or under the influence of Aph would be expected to be lower than those induced with unfixed APCs in the absence of Aph. Nonetheless, recall responses from these populations are comparable with those cells primed only with irradiated APCs. This suggests that, in addition to inhibiting primary proliferation, these manipulations may have actually enhanced the capability of the resultant primed T cells to respond to recall stimuli. Cell proliferation and differentiation are frequently not simultaneous events. For example, restricting proliferation of B cells by Aph leads to enhanced frequencies IgA production in clonal B cell microcultures. It is possible that commitment to memory vs effector T cell pathways may be similarly regulated. If the stimulus results in rapid cell replication, a strong effector response and a relatively weaker memory response may develop, while a weaker stimulus may lead to lesser effector responses immediately, but better recall responses later. Thus, while the infectious load is large, for example, an immediate effector response is needed for early clearance of the invaders and as the antigenic load declines, the number of effectors required will decrease while the memory compartment would need to be primed for future eventualities.

Such possibilities make it plausible that signal-transduction pathways can be identified that differentially affect the commitment of responding T cells to the effector vs the memory pathways. IL-2 is the major driver for proliferation of T cells, especially for naive T cells since our data to date suggest that proliferation is not necessary for the induction of efficient memory. It was of interest to look at the role of IL-2 in these events. We chose two drugs that are known to inhibit IL-2 transcription and T cell proliferation through different pathways. Trental and CsA, Trental at 100 μg/ml causes significant inhibition of the primary proliferative response (FIG. 7A), and we do not find detectable levels of IL-2 in culture supernatants from such Trental-treated cultures (data not shown). Yet, the recall responses of cells primed under cover of Trental are far higher than those of cells primed without Trental (FIG. 7B). In fact, if Trental is used in a situation in which T cell proliferation is abrogated by Aph it still enhances the response capability of primed T cells (FIG. 9). The experiments following the kinetics of the primary response show that the drugs used do not cause any delay in the fully activation of the primary response, even if they are removed at day 6 (FIG. 10) implying that the enhanced recall responses measured do indeed represent secondary or memory like responses rather simply altered primary responses. The enhancement in the magnitude of the secondary response caused by the presence of Trental during priming is not simply the result of altered kinetics or response either (FIG. 11), but an actual increase. Clearly Trental affects T cell priming such that commitment to memory is enhanced at the cost of effector responses, independent of its on T cell proliferation. The quality of memory responses generated in terms of the cytokine profile, does not appear to be affected significantly by Trental, since the levels of IFN-γ produced during recall are not different between cells primed in the presence of abeyance of Trental (data not shown). This is interesting in light of reports that Trental preferentially inhibits the activation of Th1 cells.

Trental is a phosphodiesterase inhibitor and increases intracellular concentrations of cAMP. It is also known to inhibit the induction of the c-rel family of proteins possibly involved in the transcriptional regulation of both the IL-2 and the IL-2R genes. Transcription of IL-2 is inhibited clearly by Trental, although there is some uncertainty about its effects on induction of high affinity IL-2R. High concentrations of cAMP also inhibit the induction of IL-2 gene transcription factors and if IL-2 transcription; again it is not clear whether the effects of Trental are mediated through this pathway. CsA as an inhibitor of calcineurin inhibits the induction of the IL-2 gene transcription factors and of IL-2 transcription. Again it is not clear whether the effects of Trental are mediated through this pathway. CsA as an inhibitor of calcineurin inhibits the induction of the IL-2 transcription factor. Nuclear Factor of Activated T cells (NF-AT) (28), causing blockade of IL-2 transcription. A drug closely related to CsA FK506 is knows to inhibit the induction of at least some members of the c-rel family problems as well, but the identities of the specific members affected are different from those modulated by Trental. It was thus of interest to examine the effects of CsA in the present experimental system and to compare them with those of Trental.

All doses of Csa cause significant inhibition of the primary proliferative response, as expected (FIG. 12A). Relatively high doses of CsA used during priming, however, still permit priming, although they cause a decrease in the recall response below that of responders primed in the absence of the drug. At a low dose 0.1 μg/ml, CsA causes an enhancement of recall capacity reminiscent of that caused by Trental. Trental, however does not induce any reduction in memory commitment at any concentration, and its effects on recall enhancement increase over the dose range used (FIG. 8). The two drugs may therefore differently affect pathways relevant to immune memory.

The presence of anti-IL-2 Abs or of CsA (1 μg/ml) during T cell priming has been reported previously to cause enhancement in the commitment to T cell memory, suggesting that removal of IL-2 during priming leads to a referential commitment to T cell memory, although the readout used was an effector function assay for B cell help rather than a recall of memory. Our data do show that T cell priming is perfectly possible in the presence of CsA, but they also demonstrate that only low doses permit actual enhancement. The induction of T cell memory is unlikely, in any event to be correlated in a simple fashion to IL-2 availability alone since if that were true, the dose-response relationship of the effect of CsA on memory commitment would not have been a negative one. Unlike FK506 and presumably, CsA, Trental does not inhibit the induction of the IL-2 transcription factor Nuclear Factor of Activated T cells but all of these drugs appear to inhibit the induction of some members at least of the c-rel family. It is therefore possible that commitment to T cell effector vs. memory pathways is dependent on some balance of the transcription factors that are differently affected by these drugs. The pathway blockade of IL-2 transcription may in fact be more relevant for the consequences of priming than simply the presence or absence of IL-2.

In addition, the absence of co-stimulatory signals, especially the CD28-B7 interaction has been demonstrated to cause T cell anergy and the effects of this interaction appear also to be on IL-2 transcription. We have shown previously that when activated T cells are used as APCs for priming. There are no detectable levels of IL-2 observed nor any primary proliferation, but there is induction of Ag-specific tolerance instead of the enhancement of recall capacity seen in this study with Trental. Thus, it is also possible that the role of co-stimulatory signals in regulating commitment of the responding T cells to anergy vs. memory pathways may involve effects other than on the transcription of IL-2. In this context, it is interesting to note that Trental-mediated enhancement in priming is unaffected by the presence of exogenous IL-2 (data not shown) once again suggesting that IL-2 limitation is not the pathway by which Trental enhances priming.

CsA has been used extensively as a therapeutic immunosuppressant in clinical transplantation even though CsA is very effective in suppressing the primary response, it may still permit T cell priming and this is an issue that needs to be kept in mind in both identifying and using immunosuppressants.

Whereas, the present invention has been described in relation to the drawings attached hereto, it should be understood that other and further modifications, apart from those shown or suggested herein, may be made within the spirit and scope of this invention.

What is claimed is:

1. A vaccine for inducing immune T lymphocytes to respond to a target antigen at first exposure such that they respond to a greater magnitude upon any subsequent exposure to the same antigen, the vaccine consisting essentially of antigen and a therapeutically effective quantity of pentoxifylline.

2. A vaccine, for inducing immune T lymphocytes to respond to a target antigen at first exposure such that they respond to a greater magnitude upon any subsequent exposure to the same antigen, the vaccine consisting essentially of pentoxifylline, and at least one antigen or immunogen, wherein the pentoxifylline is in an amount usable to provide a dose of 10 mg to 100 mg per kg body weight.

3. The vaccine of claim 2, further comprising a PBS, saline, or distilled water carrier.

4. The vaccine of claim 3, further comprising an alum immunoadjuvant.

5. The vaccine of claim 2, further comprising at least one vitamin or mineral immune system booster.

6. In a vaccine, the improvement comprising a pentoxifylline immunoadjuvant wherein the immunoadjuvant reduces the primary T cell response and enhances the antigen-specific T cell immune memory, wherein the pentoxifylline is an amount usable to provide a dose of 10 mg to 100 mg per kg body weight.

7. An improved vaccine, comprising:
an otherwise poor immunogen, and pentoxifylline.

8. A vaccine consisting essentially of antigen and a therapeutically effective quantity of pentoxifylline, wherein the pentoxifylline is administered at least one of before, during and after administration of the antigen.
